(19) United States
(12) Patent Application Publication
(10) Pub. No.: US 2020/0312431 A1
(43) Pub. Date: Oct. 1, 2020

(54) METHOD, SYSTEM, AND APPARATUS FOR AUTOMATICALLY ADDING ICD CODE, AND MEDIUM

(71) Applicant: BOE TECHNOLOGY GROUP CO., LTD., Beijing (CN)

(72) Inventors: Zhenzhong ZHANG, Beijing (CN); Xue CHEN, Beijing (CN)

(73) Assignee: BOE TECHNOLOGY GROUP CO., LTD., Beijing (CN)

(21) Appl. No.: 16/710,193

(22) Filed: Dec. 11, 2019

(30) Foreign Application Priority Data
Mar. 29, 2019 (CN) 201910251696.8

(51) Int. Cl. G16H 10/60; G16H 70/60

(52) U.S. Cl. CPC G16H 10/60; G16H 70/60

(57) ABSTRACT

The disclosure provides a method, system, and apparatus for automatically adding ICD code, and a medium. The method for automatically adding an ICD (International Classification of Diseases) code includes: obtaining medical record data; obtaining a vector representation of a respective medical record element through the medical record data; obtaining a vector representation of a respective disease in ICD; obtaining, based on the vector representation of the respective medical record element and the vector representation of the respective disease in ICD, a vector representation of medical record; obtaining, based on the vector representation of the medical record, a respective probability of adding a respective disease code in ICD to the medical record; and obtaining, based on the respective probability, an indication of whether to automatically add the respective ICD code in ICD corresponding to the respective probability to the medical record.

### PRIORITY

In accordance with the applicable Patent Law and/or the Paris Convention, this application is intended to promptly request the priority and benefit of the application with the application number 201910251696.8 filed on Mar. 29, 2019.

### TECHNICAL FIELD

The present application relates to the medical field, and more particularly, to a method, system, and apparatus for automatically adding ICD code, and a medium.

### BACKGROUND

International Classification of Diseases (ICD10) is an internationally unified classification of diseases for various diseases according to certain characteristics of diseases and based on rules. Assigning the correct ICD10 code to each patient (that is, adding the ICD10 code to the patient's medical record) based on the diagnosis is important for clinical application and management. However, assigning the correct. ICD10 code to the patient at the time of doctor visiting requires a lot of manpower, material and financial resources. For example, statistics show that the annual financial expenditure in the United States to improve the quality of code is as high as $25 billion. In addition, when assigning codes, the medical code staff needs to consult the doctor's diagnosis described using text phrases and sentences and other information in the electronic medical record, and then to manually assign the appropriate ICD10 code according to the Code Guide, in which process it is easy for a variety of errors to appear. For example, doctors often use abbreviations and synonyms when writing diagnostic descriptions, which can lead to confusion and inaccuracy when the code staff match ICD10 codes with these abbreviations and synonyms.

In addition, in many cases, multiple diagnostic descriptions are closely related and should be combined into a single combined ICD10 code. However, the ICD10 code is organized in a hierarchical structure in which the upper layer code represents a broad range of disease category and the lower layer code represents a more specific disease. Therefore, when the code staff matches the diagnostic descriptions to an overly broad code, rather than a more specific code, miscoding can also occur.

### SUMMARY OF THE INVENTION

According to one aspect of the present disclosure, a method for automatically adding an ICD (International Classification of Diseases) code is provided, comprising: obtaining medical record data; obtaining a vector representation of a respective medical record element through the medical record data; obtaining a vector representation of a respective disease in ICD; obtaining, based on the vector representation of the respective medical record element and the vector representation of the respective disease in ICD, a vector representation of medical record; obtaining, based on the vector representation of the medical record, a respective probability of adding a respective disease code in ICD to the medical record; and obtaining, based on the respective probability, an indication of whether to automatically add the respective ICD code in ICD corresponding to the respective probability to the medical record.

According to one aspect of the present disclosure, wherein obtaining a vector representation of a respective medical record element through the medical record data comprises: obtaining a vector representation of a respective medical record element through a word vector of the respective medical record element contained in the medical record data.

According to one aspect of the present disclosure, wherein obtaining a vector representation of a respective disease in ICD comprises: obtaining a vector representation of a respective disease in ICD through a word vector of the respective disease in ICD

According to one aspect of the present disclosure, wherein obtaining, based on the vector representation of the respective medical record element and the vector representation of the respective disease in ICD, a vector representation of medical record comprises: obtaining, based on the vector representation of the respective medical record element and the vector representation of the respective disease in ICD, a contribution of the vector representation of the respective medical record element to the vector representation of the respective disease; and obtaining, based on the contribution of the vector representation of the respective medical record element to the vector representation of the respective disease, a vector representation of medical record.

According to one aspect of the present disclosure, wherein a contribution of the vector representation d of the respective medical record element to the vector representation iₖ of a k-th disease is expressed as:

aₖ=soft max(d₁^{T}iₖ, d₂^{T}iₖ, d₃^{T}iₖ, . . . dₙ^{T}iₖ)

$$max ( x ) = \frac{exp ( x )}{{Σ}_{j} exp ( {x}_{j} )} ,$$

where soft x is a vector, xⱼ is a j-th element in the vector x, dₙ^{T} denotes the transposition of dₙ, and d₁, d₂, d₃ . . . dₙ respectively denote the vector representation of the respective medical record element.

According to one aspect of the present disclosure, wherein obtaining, based on the respective probability, an indication of whether to automatically add the respective ICD code in ICD corresponding to the respective probability to the medical record comprises: if the probability is greater than a predetermined threshold, adding the disease code in ICD corresponding to the probability to the medical record; if the probability is less than a predetermined threshold, not adding the disease code in ICD corresponding to the probability to the medical record.

According to one aspect of the present disclosure, wherein the, probability of adding the k-th disease code in ICD to the medical record based on the vector representation of the medical record is expressed as:

yₖ=σ(βₖ^{T}·dₖ+bₖ)

where σ( ) is a sigmoid function, βₖ and bₖ are linear parameters obtained through neural network training, dₖ denotes the vector representation of the medical record.

According to one aspect of the present disclosure, wherein the medical record element includes one or more elements related to medical record data among chief complaint, history of present illness, and history of past illness.

According to another aspect of the present disclosure, a system for automatically adding an ICD (International Classification of Diseases) code is provided, comprising: a medical record data obtaining module for obtaining medical record data; a vector-representation-of-medical-record-element obtaining module for obtaining a vector representation of a respective medical record element through the medical record data; a vector-representation-of-disease obtaining module for obtaining a vector representation of a respective disease in ICD; a vector-representation-of-medical-record obtaining module for obtaining, based on the vector representation of the respective medical record element and the. vector representation of the respective disease in ICD, a vector representation of medical record; a probability obtaining module for obtaining, based on the vector representation of the medical record, a respective probability of adding a respective disease code in ICD to the medical record; and an adding module for obtaining, based on the respective probability, an indication of whether to automatically add the respective ICD code in ICD corresponding to the respective probability to the medical record.

According to yet aspect of the present disclosure, an apparatus for automatically adding an ICD (International Classification of Diseases) code is provided, comprising: a processor; and a memory in which computer-readable instructions are stored, wherein a method for automatically adding an ICD code is performed when the computer-readable instructions are executed by the processor, said method comprising: obtaining medical record data; obtaining a vector representation of a respective medical record element through the medical record data; obtaining a vector representation of a respective disease in ICD; obtaining, based on the vector representation of the respective medical record element and the vector representation of the respective disease in ICD, a vector representation of medical record; obtaining, based on the vector representation of the medical record, a respective probability of adding a respective disease code in ICD to the medical record; and obtaining, based on the respective probability, an indication of whether to automatically add the respective ICD code in ICD corresponding to the respective probability to the medical record.

According to yet another aspect of the present disclosure, a computer-readable storage medium for storing a computer-readable program is provided, the program causing a computer to perform the method for automatically adding an ICD code.

### BRIEF DESCRIPTION OF THE DRAWINGS

Through the more detailed description of embodiments of the present disclosure. with reference to the accompanying drawings, the above and other objectives, features, and advantages of the present disclosure will become more apparent. The drawings are to provide further understanding for the embodiments of the present disclosure and constitute a portion of the specification, and are intended to interpret the present disclosure together with the embodiments rather than to limit the present disclosure. In the drawings, the same reference sign generally refers to the same component or step.

FIG. 1 shows steps of a method for automatically adding an ICD code in accordance with an embodiment of the present disclosure.

FIG. 2 shows a schematic diagram of obtaining a vector representation of a medical record element in accordance with an embodiment of the present disclosure.

FIG. 3 shows steps of obtaining a vector representation of a medical record in accordance with an embodiment of the present disclosure.

FIG. 4 shows a block diagram of a structure for automatically adding an ICD code in accordance with an embodiment of the present disclosure.

FIG. 5 shows a schematic diagram of a system for automatically adding an ICD code in accordance with an embodiment of the present disclosure.

FIG. 6 shows a schematic diagram of an apparatus for automatically adding an ICD code in accordance with an embodiment of the present disclosure.

### DESCRIPTION OF THE EMBODIMENTS

The technical solutions in the embodiments of the present disclosure will be described in a clear and comprehensive way with reference to the accompanying drawings thereof. Obviously, the described embodiments are only part of the embodiments of the present disclosure, rather than all of the embodiments of the present disclosure, it should be understood that the present disclosure is not limited to the exemplary embodiments described herein. All other embodiments obtained by a person of ordinary skill in the art without paying inventive efforts should fall into the, protection scope of the present disclosure.

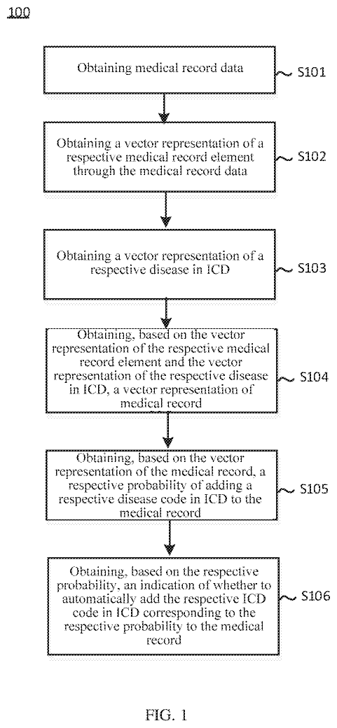

FIG. 1 shows steps of a method for automatically adding an ICD (ICD10) code in accordance with an embodiment of the present disclosure. The method can be automatically completed by a computer or the like without manual participation, thereby it can effectively save manpower and financial resources, improve efficiency and user experience.

As shown in FIG. 1, in step S101, medical record data is obtained.

For example, the medical record data may be data related to the user's current disease, such as data related to the current disease from among chief complaint, history of present illness, and history of past illness of a given medical record, as well as a diagnosis result given by a doctor, and the like.

In the embodiment of the present disclosure, the medical record may be a handwritten medical record, data required in the medical record is obtained by OCR or manual reading; or the medical record may be an electronic medical record, required data may be derived through a management platform of the electronic medical record.

In step S102, a vector representation of a respective medical record element is obtained through the medical record data.

For example, the medical record element may include one or more elements related to medical record data among chief complaint, history of present illness, and history of past illness. For example, the medical record element may be denoted as a chief complaint element, a history-of-present-illness element, and a history-of-past-illness element.

As an example, obtaining a vector representation of a respective medical record element through the medical record data may comprise: obtaining a vector representation of a respective medical record element through a word vector of the respective medical record element contained in the medical record data. The vector representation of the medical record element may be obtained via a neural network.

The neural network is a large-scale, multi-parameter optimization tool. With a large amount of training data, a deep neural network can learn hidden features that are difficult to summarize in data, thus completing multiple complex tasks such as face detection, image semantic segmentation, text abstract extraction, object detection, motion tracking, natural language translation, etc. For example, obtaining a vector representation of a text through a word vector refers to representing each word in the text as a single vector, and generating a vector representation of the text by performing a high degree of summarization and abstraction. There are many methods for generating a vector representation of a text through a word vector using a neural network, details will not be described herein.

FIG. 2 shows a schematic diagram of obtaining a vector representation of a medical record element in accordance with the embodiment of the present disclosure. As shown in FIG. 2, for example, the word vector of a respective word in the chief complaint element is denoted as c1 (21 in FIG. 2), c2 (22 in FIG. 2) . . . cn (23 in FIG. 2), and then these word vectors are used as input, and the vector representation d₁ 25 of the chief complaint element can be obtained by a neural network 24. Similarly, vector representations d₂ and d₃ (not shown) of the history-of-present-illness element and the history-of-past-illness element can be obtained by this method. It should be understood that the manner of obtaining the vector representation of the respective medical record element is not limited thereto, the vector representation of the respective medical record element may also be obtained by other means. It should be understood that the medical record elements are not limited to the above-mentioned three types of chief complaint, history of present illness, history of past illness. Further, for example, it is also possible to uniformly denote the vector of the medical record element as dₙ, where n is a natural number.

For example, the neural network 24 may be a GRU (Gated Cycle Unit) network with a word vector as input, a vector representation of the corresponding content is obtained through a GRU (Gated Cycle Unit) network, and an output of a last neural unit of the GRU network is a vector representation of the medical record element.

Although an implementation of the neural network 24 is described above using the GRU as an example, other neural networks, such as a Recurrent Neural Network (RNN), a Long Short Term Memory (LSTM), a Bidirectional Recurrent Neural Network (BiRNN), Simple Recurrent Unit (SRU) etc. may also be applicable.

Although not specifically described, a person skilled in the art will readily appreciate that the above functions can be performed by selecting a neural network obtained through sample medical record training.

Next, returning to FIG. 1, in step S103, a vector representation of a respective disease in ICD10 is obtained.

As an example, obtaining the vector representation of the respective disease in ICD10 may comprise: obtaining the vector representation of the respective disease in ICD10 through the word vector of the respective disease in ICD10. For example, the vector representation of the respective disease can be obtained by the method shown in FIG. 2, details are not described herein again. It should be understood that the manner of obtaining the vector representation of the respective disease in ICD10 is not limited thereto, the vector representation of the respective disease in ICD10 may also be obtained by other means.

Next, in step S104, a vector representation of medical record is obtained based on the vector representation of the respective medical record element and the vector representation of the respective disease in ICD10.

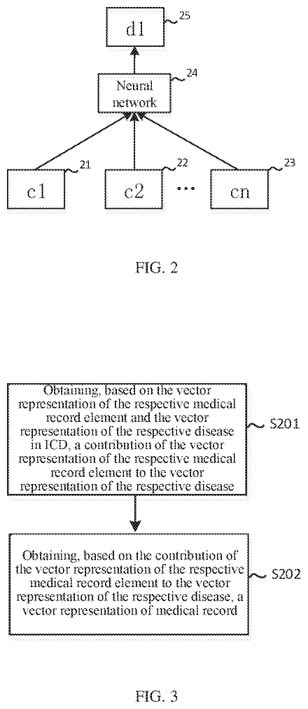

FIG. 3 shows steps of obtaining a vector representation of a medical record in accordance with the embodiment of the present disclosure. As can be seen from FIG. 3, obtaining, based on the vector representation of the respective medical record element and the vector representation of the respective disease in ICD10, the vector representation of medical record comprises two steps: obtaining (S201), based on the vector representation of the respective medical record element and the vector representation of the respective disease in ICD10, a contribution of the vector representation of the respective medical record element to the vector representation of the respective disease; and obtaining (S202), based on the contribution of the vector representation of the respective medical record element to the vector representation of the respective disease, a vector representation of medical record.

For example, the vector representation of the medical record may be obtained in accordance with the following method.

First, a vector representation of a k-th disease (1≤k≤V, V denotes a total number of diseases in ICD10) in the given ICD10 is calculated through step S103 in FIG. 1 as iₖ.

Thereafter, a contribution aₖ of the vector representation of the respective medical record element (such as a chief complaint element d₁, a history-of-present-illness element d₂, a history-of-past-illness element d₃) to the vector representation of the respective disease in ICD10 is expressed as:

aₖ=soft max(dₗ^{T}iₖ, d₂^{T}iₖ, d₃^{T}iₖ, . . . d₃^{T}iₖ) (1)

where soft

$$max ( x ) = \frac{exp ( x )}{{Σ}_{j} exp ( {x}_{j} )} ,$$

x is a vector, xⱼ is a j-th element in the vector x, dₙ^{T} denotes the transposition of dₙ.

Then based on the contribution aₖ, the vector representation of the medical record may be represented as:

dₖ=Σₘ₌₁³a_{k,m}dₘ (2)

where a scalar a_{k,m}, denotes an m-th element of the contribution vector aₖ, dₘ(m=1, 2, 3 . . . n) denotes the vector representation of the element dₙ such as a chief complaint element d₁, a history-of-present-illness element d₂, a history-of-past-illness element d₃.

Thereafter, returning to FIG. 1, in step S105, a respective probability of adding a respective disease code in ICD10 to the medical record is obtained based on the vector representation of the medical record.

For example, the vector representation of the medical record is taken as the input, a respective probability of adding a respective disease code in ICD10 to the medical record may be obtained through a classifier, the specific method is as follows:

Yₖ=σ(βₖ^{T}·dₖ+bₖ) (3)

where σ( ) is a sigmoid function, βₖ is a parameter vector having the same dimension as dₖ, and bₖ is a scaler, βₖ and bₖ are both obtained through the aforesaid neural network training.

In the neural network, the above system parameters (βₖ and bₖ) can be learned by minimizing the following function (for example, the cross entropy loss function) by using a random gradient descent method:

L=−Σₚ₌₁^{p}lₚlog(yₚ)+(1−lₚ)log(1−yₚ) (4)

where P denotes a total number of training data in the neural network, p denotes a p-th training data (1≤p≤P), lₚ denotes a probability (expected output) of adding the ICD10 code of the p-th training data to the medical record, yₚ denotes the probability (actual output) of adding the ICD10 code of the p-th training data as predicted by the system in the neural network to the medical record.

Alternatively, the respective probability of adding the respective disease code in ICD10 to the medical record can also be obtained by the following formula:

$${y}_{k} ( {s}_{k} \vert T ) = \frac{1}{z ( T )} exp ( {\sum }_{q = 1}^{Q} {w}_{q} {f}_{q} ( T , {s}_{k} ) ) \qquad ( 5 )$$

where T denotes a given medical record, sₖ denotes the ICD10 code of a k-th disease, w_{q} is a parameter obtained from training (for example, obtained by the above similar training method), f_{q}(T, sₖ) denotes a q-th (1≤q≤Q, Q denotes a total number of feature values) feature value obtained based on the given medical record. T and the ICD10 code of the k-th disease, f_{q}(T, sₖ)=d_{k,q}, d_{k,q} denotes a q-th element of the vector representation dₖ of the medical record, z(T) denotes a normalization factor (which ensures that the sum of probabilities is one), wherein

z(T)=Σₛ_{ₖ}exp (Σ_{q=1}^{Q}w_{q}f_{q}(T, sₖ)) (6)

It should be appreciated that the above-described method for obtaining the respective probability of adding the respective disease code in ICD10 to the medical record is not limited thereto, and the respective probability of adding the respective disease code in ICD10 to the medical record may be obtained also by other methods.

Next, returning to FIG. 1, in step S106, an indication of whether to automatically add the respective ICD code in ICD10 corresponding to the respective probability to the medical record is obtained based on the respective probability.

For example, if the probability yₖ is greater than a predetermined threshold, the code in ICD10 of the disease k corresponding to the probability yₖ is added to the medical record; if the probability yₖ is less than the predetermined threshold, the code of the disease k in ICD10 corresponding to the probability yₖ is not added to the medical record. The predetermined threshold may be preset or specified in advance according to statistical laws. For example, the predetermined threshold may be set to 0.5. Assuming that a medical record is given, when the probability of adding the code (G70.001) for myasthenia gravis to the medical record is greater than or equal to 0.5 by the method described above, the code G70.001 is added to the given medical record, otherwise it is not added to the given medical record.

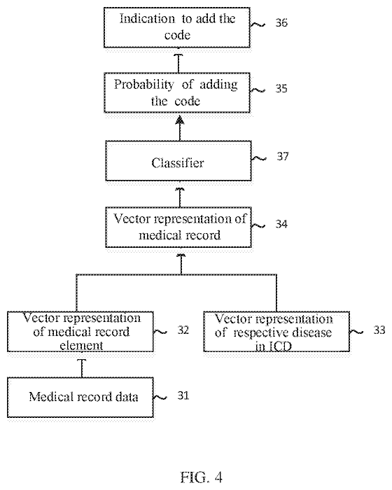

FIG. 4 shows a block diagram of a structure for automatically adding an ICD10 code in accordance with an embodiment of the present disclosure. After the above method for automatically adding an ICD10 code in accordance with an embodiment of the present disclosure is described in detail with reference to FIGS. 1-3, for the sake of clarity, a method for automatically adding an ICD10 code in accordance with an embodiment of the present disclosure will be briefly described below with reference to FIG. 4.

As shown in FIG. 4, in order to effectively save manpower and financial resources and to improve efficiency and user experience, the present disclosure provides a method for automatically adding an ICD10 code. In this method, medical record data 31 is first obtained, thereafter a vector representation 32 of the respective medical record element is obtained by using the neural network based on the medical record data 31. At the same time, a neural network is used to obtain a vector representation 33 of the respective disease in ICD10. After the vector representation 31 of the respective medical record element and the vector representation 33 of the respective disease in ICD10 are obtained, a vector representation 34 of the medical record is obtained based on both of them. Then a probability 35 of automatically adding the ICD10 code is obtained by a classifier 37 with the use of the vector representation 34, and finally an indication 36 of whether to automatically add the ICD10 code is obtained based on the probability.

In the above aspect of the present disclosure, the method for automatically adding the ICD code is provided. Specifically, the present disclosure obtains a vector representation of a medical record through the neural network based on medical record data and the respective disease description in ICD, and then generates the indication of whether to automatically add the ICD code based on the vector representation of the medical record, accordingly, human and financial resources are effectively saved, efficiency and user experience are improved, and a database of various medical records can also be created to provide a basis for future medical record research.

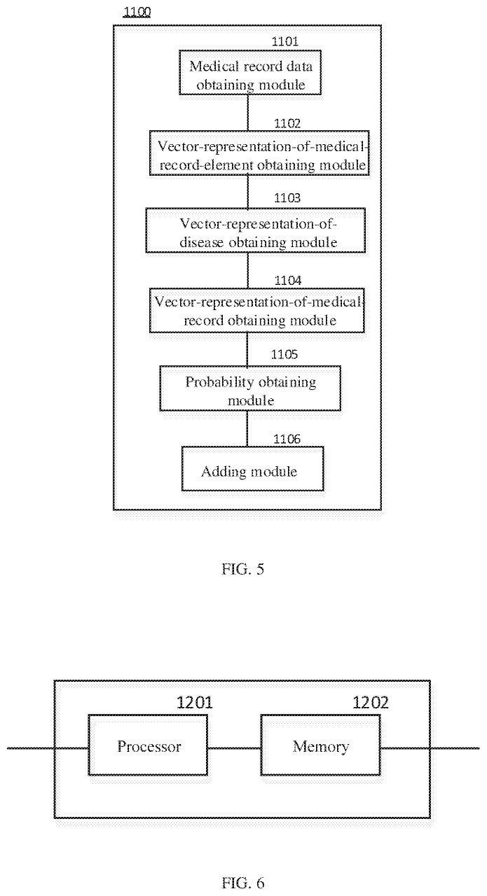

A system 1100 for automatically adding an ICD10 code in accordance with an embodiment of the present disclosure is described below with reference to FIG. 5. FIG. 5 is a schematic diagram of a system 1100 for automatically adding an ICD10 code in accordance with an embodiment of the present disclosure. Since the functions of the system for automatically adding an ICD10 code of the present embodiment are the same as the details of the method 100 described above with reference to FIG. 1, detailed description of the same contents are omitted herein for the sake of simplicity.

As shown in FIG. 5, the system 1100 for automatically adding an ICD10 code comprises a medical record data obtaining module 1101, a vector-representation-of-medical-record-element obtaining module 1102, a vector-representation-of-disease obtaining module 1103, a vector-representation-of-medical-record obtaining module 1104, a probability obtaining module 1105, and an adding module 1106. It should be noted that although the system 1100 for automatically adding the: ICD10 code in FIG. 5 is shown to comprise only six modules, this is only illustrative, the system 1100 for automatically adding the ICD10 code may also comprise one or more other modules, these modules are not related to the inventive concept and are therefore omitted herein.

In the present disclosure, the medical record data obtaining module 1101 is used for obtaining medical record data. The vector-representation-of-medical-record-element obtaining module 1102 is for obtaining a vector representation of a respective medical record element through the medical record data.

For example, the medical record element may include one or more elements related to medical record data from among chief complaint, history of present illness, and history of past illness. For example, the medical record element may be denoted as a chief complaint element, a history-of-present-illness element, and a history-of-past-illness element.

As an example, obtaining the vector representation of the respective medical record element through the medical record data may comprise: obtaining the vector representation of the respective medical record element through a word vector of the respective medical record element contained in the medical record data. The vector representation of the medical record element may be obtained via a neural network. The concept of the neural network has been described in detail in the above, method steps will not be described again herein.

FIG. 2 shows a schematic diagram of obtaining a vector representation of a medical record element in accordance with the embodiment of the present disclosure. As shown in FIG. 2, for example, the word vector of a respective word in the chief complaint element is denoted as c1 (21 in FIG. 2), c2 (22 in FIG. 2) . . . cn (23 in FIG. 2), then these word vectors are used as input, and the vector representation d₁ 25 of the chief complaint element can be obtained by a neural network 24. Similarly, vector representations d₂ and d₃ (not shown) of the history-of-present-illness element and the history-of-past-illness element can be obtained by this method. It should be understood that the manner of obtaining the vector representation of the respective medical record element is not limited thereto, the vector representation of the respective medical record element may also be obtained by other means. It should be understood that the medical record elements are not limited to the above-mentioned three types of chief complaint, history of present illness, history of past illness. Further, for example, it is also possible to uniformly denote the vector of the medical record element as dₙ, where n is a natural number.

Next, the vector-representation-of-disease obtaining module 1103 is for obtaining the vector representation of a respective disease in ICD10.

As an example, obtaining the vector representation of the respective disease in ICD10 may comprise: obtaining the vector representation of the respective disease in ICD10 through the word vector of the respective disease in ICD10. For example, the vector representation of the respective disease can be obtained by the method shown in FIG. 2, details are not described herein again. It should be understood that the manner of obtaining the vector representation of the respective disease in ICD10 is not limited thereto, the vector representation of the respective disease in ICD10 may also be obtained by other means.

Next, the vector-representation-of-medical-record obtaining module 1104 is for obtaining, based on the vector representation of the respective medical record element and the vector representation of the respective disease in ICD10, a vector representation of medical record.

FIG. 3 shows steps of obtaining a vector representation of a medical record in accordance with the embodiment of the present disclosure. As can be seen from FIG. 3, obtaining, based on the vector representation of the respective medical record element and the vector representation of the respective disease in ICD10, the vector representation of medical record comprises two steps: obtaining (S201), based on the vector representation of the. respective medical record element and the vector representation of the respective disease in ICD10, a contribution of the vector representation of the respective medical record element to the vector representation of the respective disease; and obtaining (S202), based on the contribution of the vector representation of the respective medical record element to the vector representation of the respective disease, a vector representation of medical record.

For example, the vector representation of the medical record may be obtained in accordance with the following method.

First, the vector-representation-of-disease obtaining module 1103 calculates a vector representation of a k-th disease (1≤k≤V, V denotes a total number of diseases in ICD10) in the given ICD10 as iₖ.

Thereafter, a contribution aₖ of the vector representation of the respective medical record element (a chief complaint element d₁, a history-of-present-illness element d₂, a history--of-past-illness element d₃) to the vector representation of the respective disease in ICD10 is expressed as:

aₖ=soft max(d₁^{T}iₖ, d₂^{T}iₖ, d₃^{T}ₖ, . . . dₙ^{T}iₖ)

where soft

$$max ( x ) = \frac{exp ( x )}{{Σ}_{j} exp ( {x}_{j} )} ,$$

x is a vector, xⱼ is a j-th element in the vector x, dₙ^{T} denotes the transposition of dₙ. In the present disclosure, the letters indicated in bold represent vectors.

Then, based on the contribution aₖ, the vector representation of the medical record may be represented as:

$${d}_{k} = {\sum }_{m = 1}^{3} {a}_{k , m} {d}_{m}$$

where a scalar a_{k,m} denotes an m-th element of the contribution vector aₖ, dₘ(m=1, 2, 3 . . . n) denotes the vector representation of the element dₙ such as a chief complaint element d₁, a history-of-present-illness element d₂, a history-of-past-illness element d₃.

Thereafter, the probability obtaining module 1105 is for obtaining, based on the vector representation of the medical record, a respective probability of adding a respective disease code in ICD10 to the medical record.

For example, the vector representation of the medical record is taken as the input, a respective probability of adding a respective disease code in ICD10 to the medical record may be obtained through a classifier, the specific method is as follows:

yₖ=σ(βₖ^{T}·dₖ+bₖ)

where σ( ) is a sigmoid function, βₖ is a parameter vector having the same dimension as dₖ, and bₖ is a scaler, βₖ and bₖ are both obtained through the aforesaid neural network training.

In the neural network, the above system parameters (βₖ and bₖ) can be learned by minimizing the following function (for example, the cross entropy loss function) by using a random gradient descent method:

$$L = - {\sum }_{p = 1}^{P} {l}_{p} log ( {y}_{p} ) + ( 1 - {l}_{p} ) log ( 1 - {y}_{p} )$$

where P denotes a total number of training data in the neural network, p denotes a p-th training data (1≤p≤P), lₚ denotes a probability (expected output) of adding the ICD10 code of the p-th training data to the medical record, yₚ denotes the probability (actual output) of adding the ICD10 code of the p-th training data as predicted by the system in the neural network to the medical record.

Alternatively, the respective probability of adding the respective disease code in the ICD10 to the medical record can also be obtained by the following formula:

$${y}_{k} ( {s}_{k} \vert T ) = \frac{1}{z ( T )} exp ( {\sum }_{q = 1}^{Q} {w}_{q} {f}_{q} ( T , {s}_{k} ) ) \qquad ( 5 )$$

where T denotes a given medical record, sₖ denotes the ICD10 code of a k-th disease, w_{q} a parameter obtained from training (for example, obtained by the above similar training method), f_{q}(T,sₖ) denotes a q-th (1≤q≤Q, Q denotes a total number of feature values) feature value obtained based on the given medical record T and the ICD10 code of the k-th disease, f_{q}(T, sₖ)=d_{k,q}, d_{k,q} denotes a q-th element of the vector representation dₖ of the medical record, z(T) denotes a normalization factor (which ensures that the sum of probabilities is one), wherein

z(T)=Σₛ_{ₖ}exp(Σ_{q=1}^{Q}w_{q}f_{q}(T, sₖ)) (6)

It should be appreciated that the above-described method for obtaining the respective probability of adding the respective disease code in ICD10 to the medical record is not limited thereto, the respective probability of adding the respective disease code in ICD10 to the medical record may be obtained also by other methods.

Next, the adding module 1106 is for obtaining, based on the respective probability, an indication of whether to automatically add the respective ICD10 code in ICD10 corresponding to the respective probability to the medical record.

For example, if the probability yₖ is greater than a predetermined threshold, the code in ICD10 of the disease k corresponding to the probability yₖ is added to the medical record; if the probability yₖ is less than the predetermined threshold, the code of the disease k in ICD10 corresponding to the probability yₖ is not added to the medical record. The predetermined threshold may be preset (for example, the predetermined threshold may be set to 0.5) or specified in advance according to statistical laws.

In the above-described embodiments, the description manner of the functional modules corresponding to the functions to be performed is used, and it is easy to understand that these modules are functional entities and do not necessarily have to correspond to entities that are physically or logically independent. These functional entities may be implemented by running software in the form of executing computer instructions, or programmably implemented in one or more hardware or integrated circuits.

An apparatus 1200 for automatically adding an ICD10 code in accordance with an embodiment of the present disclosure will be described with reference to FIG. 6. FIG. 6 shows a schematic diagram of an apparatus for automatically adding an ICD10 code in accordance with an embodiment of the present disclosure. Since the functions of the apparatus for automatically adding the ICD10 code of this embodiment are the same as details of the method described above with reference to FIG. 1, detailed description of the same contents is omitted herein for the sake of simplicity.

As shown in FIG. 6, the apparatus 1200 for automatically adding an ICD10 code comprises a processor 1201 and a memory 1202. It should be noted that although the apparatus 1200 for automatically adding an ICD10 code in FIG. 6 is shown to comprise only two apparatuses, this is only illustrative, the apparatus 1200 for automatically adding an ICD10 code may also comprise one or more other apparatuses (such as input apparatus, display apparatus, communication apparatus, etc.), these apparatuses are not related to the inventive concept and are therefore omitted herein.

The apparatus 1200 for automatically adding the ICD10 code comprises a processor 1201; and a memory: 1202 in which computer-readable instructions are stored, wherein a method for automatically adding an ICD10 code is performed when the computer-readable instructions are executed by the processor, said method comprises: obtaining medical record data; obtaining a vector representation of a respective medical record element through the medical record data; obtaining a vector representation of a respective disease in ICD10; obtaining, based on the vector representation of the respective medical record element and the vector representation of the respective disease in ICD10, a vector representation of medical record; obtaining, based on the vector representation of the medical record, a respective probability of adding a respective disease code in ICD10 to the medical record; and obtaining, based on the respective probability, an indication of whether to automatically add the respective. ICD10 code in ICD10 corresponding to the respective probability to the medical record.

According to another aspect of the present disclosure, there is provided a computer-readable storage medium for storing a computer-readable program, the program causing a computer to perform the method for automatically adding an ICD code as described in the above.

In an embodiment of the present disclosure, the processor may be a logic computing device with data processing capabilities and/or program execution capabilities, such as a central processing unit (CPU), a field programmable logic array (FPGA), a single chip microcomputer (MCU), a digital signal processor (DSP), an application specific integrated circuit (AMC), or the like. The memory may be, for example, a volatile memory and/or a non-volatile memory. The volatile memory may include, for example, a random access memory (RAM) and/or a cache (Cache) or the like. The non-volatile memory may include, for example, a read only memory (ROM), a mechanical hard disk (HDD), a solid state drive (SSD), a flash memory (Flash), a USB flash drive, a memory card (SD, CF, MicroSD, etc.), and the like.

It will be appreciated by a person skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “data block”, “module”, “engine”, “unit,” “module,” or “system”. Furthermore, aspects of the present disclosure may take the form of a computer program product embodied in one or more computer-readable media having computer-readable program code embodied thereon.

Certain terminology has been used to describe embodiments of the present disclosure. For example, the terms “first/second embodiment”, “one embodiment”. “an embodiment”, and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having the meaning that is consistent with their meaning in the context of the relevant art and will not be interpreted in an idealized or overly formal sense unless expressly so defined herein.

The above is illustration of the present disclosure and should not be construed as making limitation thereto. Although some exemplary embodiments of the present disclosure. have been described, a person skilled in the art can easily understand that many modifications may be made to these exemplary embodiments without departing from the creative teaching and advantages of the present disclosure. Therefore, all such modifications are intended to be included within the scope of the present disclosure as defined by the appended claims. As will be appreciated, the above is to explain the present disclosure, it should not be constructed as limited to the specific embodiments disclosed, and modifications to the present disclosure and other embodiments are included in the scope of the attached claims. The present disclosure is defined by the claims and their equivalents.

What is claimed is:

1. A method for automatically adding an ICD (International Classification of Diseases) code, comprising:
obtaining medical record data;
obtaining a vector representation of a respective medical record element through the medical record data;
obtaining a vector representation of a respective disease in ICD;
obtaining, based on the vector representation of the respective medical record element and the vector representation of the respective disease in ICD, a vector representation of medical record;
obtaining, based on the vector representation of the medical record, a respective probability of adding a respective disease code in ICD to the medical record; and
obtaining, based on the respective probability, an indication of whether to automatically add the respective ICD code in ICD corresponding to the respective probability to the medical record.

2. The method for automatically adding an ICD code according to claim 1, wherein the obtaining a vector representation of a respective medical record element through the medical record data comprises:
obtaining a vector representation of a respective medical record element through a word vector of the respective medical record element contained in the medical record data.

3. The method for automatically adding an ICD code according to claim 1, wherein the obtaining a vector representation of a respective disease in ICD comprises:
obtaining a vector representation of a respective disease in ICD through a word vector of the respective disease in ICD.

4. The method for automatically adding an ICD code according to claim 1, wherein the obtaining, based on the vector representation of the respective medical record element and the vector representation of the respective disease in ICD, a vector representation of medical record comprises:
obtaining, based on the vector representation of the respective medical record element and the vector representation of the respective disease in ICD, a contribution of the vector representation of the respective medical record element to the vector representation of the respective disease; and
obtaining, based on the contribution of the vector representation of the respective medical record element to the vector representation of the respective disease, a vector representation of medical record.

5. The method for automatically adding an ICD code according to claim 4, wherein a contribution of the vector representation d of the respective medical record element to the vector representation iₖ of a k-th disease is expressed as:
aₖ=soft max(d₁^{T}iₖ, d₂^{T}iₖ, d₃^{T}iₖ, . . . dₙ^{T}iₖ)
where soft
$$max ( x ) = \frac{exp ( x )}{{Σ}_{j} exp ( {x}_{j} )} ,$$
x is a vector, xⱼ is a j-th element in the vector x, dₙ^{T} denotes the transposition of dₙ, and d₁, d₂, d₃ . . . dₙ respectively denote the vector representation of the respective medical record element.

6. The method for automatically adding an ICD code according to claim 1, wherein the obtaining, based on the respective probability, an indication of whether to automatically add the respective ICD code in ICD corresponding to the respective probability to the medical record comprises:
when the probability is greater than a predetermined threshold, adding the disease code in ICD corresponding to the probability to the medical record; when the probability is less than a predetermined threshold, not adding the disease code in ICD corresponding to the probability to the medical record.

7. The method for automatically adding an ICD code according to claim 1, wherein the probability of adding the k-th disease code in ICD to the medical record based on the vector representation of the medical record is expressed as:
yₖ=′(βₖ^{T}·dₖ+bₖ)
where σ( )is a sigmoid function, βₖ and bₖ are linear parameters obtained neural network training, dₖ denotes the vector representation of the medical record.

8. The method for automatically adding an ICD code according to claim 1, wherein the medical record element includes one or more elements related to medical record data among chief complaint, history of present illness, and history of past illness.

9. A system for automatically adding an ICD (International Classification of Diseases) code, comprising:
a medical record data obtaining module for obtaining medical record data;
a vector-representation-of-medical-record-element obtaining module for obtaining a vector representation of a respective medical record element through the medical record data;
a vector-representation-of-disease obtaining module for obtaining a vector representation of a respective disease in ICD;
a vector-representation-of-medical-record obtaining module for obtaining, based on the vector representation of the respective medical record element and the vector representation of the respective disease in ICD, a vector representation of medical record;
a probability obtaining module for obtaining, based on the vector representation of the m record, a respective probability of adding a respective disease code in ICD to the medical record; and
an adding module for obtaining, based on the respective probability, an indication of whether to automatically add the respective ICD code in ICD corresponding to the respective probability to the medical record.

10. The system for automatically adding an ICD code according to claim 9, wherein the vector-representation-of-medical-record-element obtaining module obtains a vector representation of a respective medical record element through a word vector of the respective medical record element contained in the medical record data.

11. The system for automatically adding an ICD code according to claim 9, wherein the vector-representation-of-disease obtaining module obtains a vector representation of a respective disease in ICD through a word vector of the respective disease in ICD.

12. The system for automatically adding an ICD code according to claim 9, wherein the. vector-representation-of-medical-record obtaining module obtains, based on the vector representation of the respective medical record element and the vector representation of the respective disease in ICD, a contribution of the vector representation of the respective medical record element to the vector representation of the respective disease; and
obtains, based on the contribution of the vector representation of the respective medical record element to the vector representation of the respective disease, a vector representation of medical record.

13. The system for automatically adding an ICD code according to claim 9, wherein when the probability is greater than a predetermined threshold, the adding module adds the disease code in ICD corresponding to the probability to the medical record; when the probability is less than a predetermined threshold, the adding module does not add the disease code in ICD corresponding to the probability to the medical record.

14. The system for automatically adding an ICD code according to claim 9, wherein the medical record element includes one or more elements related to medical record data among chief complaint, history of present illness, and history of past illness.

15. An apparatus for automatically adding an IC (International Classification of Diseases) code, comprising:
a processor; and
a memory in which computer--readable instructions are stored,
wherein a method for automatically adding an ICD code is performed when the computer-readable instructions are executed by the processor, said method comprising:
obtaining medical record data;
obtaining a vector representation of a respective medical record element through the medical record data;
obtaining a vector representation of a respective disease in ICD;
obtaining, based on the vector representation of the respective medical record element and the vector representation of the respective disease in ICD, a vector representation of medical record;
obtaining, based on the vector representation of the medical record, a respective probability of adding a respective disease code in ICD to the medical record; and
obtaining, based on the respective probability, an indication of whether to automatically add the respective ICD code in ICD corresponding to the respective probability to the medical record.

16. The apparatus for automatically adding an ICD code according to claim 15, wherein the. obtaining a vector representation of a respective medical record element through the medical record data comprises:
obtaining a vector representation of a respective medical record element through a word vector of the respective medical record element contained in the medical record data.

17. The apparatus for automatically adding an ICD code according to claim 15, wherein the obtaining a vector representation of a respective disease in ICD comprises:
obtaining a vector representation of a respective disease in ICD through a word vector of the respective disease in ICD.

18. The apparatus for automatically adding an ICD code according to claim 15, wherein the obtaining, based on the vector representation of the respective medical record element and the vector representation of the respective disease in ICD, a vector representation of medical record comprises:
obtaining, based on the vector representation of the respective medical record element and the vector representation of the respective disease in ICD, a contribution of the vector representation of the respective medical record element to the vector representation of the respective disease; and
obtaining, based on the contribution of the vector representation of the respective medical record element to the vector representation of the respective disease, a vector representation of medical record.

19. The apparatus for automatically adding an ICD code according to claim 18, wherein a contribution of the vector representation d of the respective medical record element to the vector representation of iₖ a k-th disease is expressed as:
aₖ=soft max(d₁^{T}iₖ, d₂^{T}iₖ, d₃^{T}iₖ, . . . dₙ^{T}iₖ)
where soft
$$max ( x ) = \frac{exp ( x )}{{Σ}_{j} exp ( {x}_{j} )} ,$$
x is a vector, x is a j-th element in the vector x, dₙ^{T} denotes the transposition of dₙ, and d₁, d₂, d₃ . . . dₙ respectively denote the vector representation of the respective medical record element.

20. A computer-readable storage medium for storing a computer-readable program, the program causing a computer to perform the method for automatically adding an ICD code as claimed in claim 1.
